(19) United States
(12) Patent Application Publication
(10) Pub. No.: US 2024/0189207 A1
(43) Pub. Date: Jun. 13, 2024

(54) COMPOSITION FOR CLEANSING AND/OR REMOVING MAKEUPS FROM KERATIN MATERIALS

(71) Applicant: L'OREAL, Paris (FR)

(72) Inventors: Lingwei XU, Shanghai (CN); Yong WANG, Shanghai (CN)

(73) Assignee: L'OREAL, Paris (FR)

(21) Appl. No.: 18/552,540

(22) PCT Filed: Jun. 24, 2021
(86) PCT No.: PCT/CN2021/102136

§ 371 Date: Sep. 26, 2023

(51) Int. Cl. A61K 8/73; A61Q 1/14

(52) U.S. Cl. CPC A61K 8/732; A61Q 1/14; A61K 2800/5422; A61K 2800/5424; A61K 2800/596

(57) ABSTRACT

A composition for cleansing and/or removing makeups from keratin materials, comprises a)at least one foaming surfactant; b)at least one nonionic cellulose selected from celluloses with at least one OH group substituted by a hydrophobic group; and c)at least one oxidized and esterified starch. A non-therapeutic process for cleansing and/or removing makeups from keratin materials, in particular the skin, comprises the application to the keratin materials, in particular the skin, of the composition, and rinsing off said composition after an optional period of time.

### TECHNICAL FIELD

The present invention relates to a cosmetic composition, in particular, a composition for cleansing and/or removing makeups from keratin materials. The present invention also relates to a non-therapeutic process for cleansing and/or removing makeups from keratin materials using said composition.

### BACKGROUD ART

Cleansing the skin or removing makeups from the skin is very important for caring for the skin. It must be as efficient as possible because greasy residues, such as excess sebum, the remnants of cosmetic products used daily and make-up products, in particular waterproof products, accumulate in the skin folds, and can block the pores of the skin and result in the appearance of spots.

Several types of skin cleansing or makeup removing products, for example, rinsable anhydrous oils and gels, foaming creams and lotions, are known.

Rinsable anhydrous oils and gels have a cleansing or makeup removing action by virtue of oils present in these formulations. These oils make it possible to dissolve fatty residues and to disperse make-up pigments. These products are effective and well tolerated. However, they exhibit the disadvantages of being heavy, of not foaming and of not conferring a good skin finish upon application, which are not desired from a cosmetic viewpoint.

Foaming creams, lotions and gels have a cleansing or makeup removing action by virtue of the surfactants, which suspend the fatty residues on the face. They are effective and pleasant to use because they foam and they are easy to remove.

Foam property is of great importance for the foaming cleanser. However, some foaming cleansers are not satisfying in terms of foam density.

In order to have better foam density in a foaming cleanser, traditional methods may include more surfactants or more polymers. However, these methods would increase the cost of formulation or bring undesired rinse-off sensory and skin finish.

Therefore, there is a need to provide another solution on improving foam density.

### SUMMARY OF THE INVENTION

The inventors have found that such a need can be achieved by the present invention.

Thus, according to an aspect, the present invention provides a composition for cleansing and/or removing makeups from keratin materials, comprising:
a) at least one foaming surfactant;
b) at least one nonionic cellulose selected from celluloses with at least one OH group substituted by a hydrophobic group; and
c) at least one oxidized and esterified starch.

The composition according to the present invention can provide a good foam density.

It may be used as a daily facial cleanser or a makeup remover. The composition of the present invention is a rinse-off product. Thus, such a composition can be applied on the skin (i.e. face and/or body), and then rinsed with flush water.

According to another aspect, the present invention provides a non-therapeutic process for cleansing and/or removing makeups from keratin materials, in particular the skin, comprising the application to the keratin materials, in particular the skin, of the composition according to the present invention, and rinsing off said composition after an optional period of time.

Other subjects and characteristics, aspects and advantages of the invention will emerge even more clearly on reading the description and the examples that follows.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values, which are capable of being changed according to the desired purpose as required.

For the purposes of the present invention, the term “keratin material” is intended to cover human skin, mucous membranes such as the lips, and the hair. Facial skin and the hair are most particularly considered according to the present invention.

All percentages in the present invention refer to weight percentage, unless otherwise specified.

According to an aspect, the present invention relates to a composition for cleansing and/or removing makeups from keratin materials, comprising:
a) at least one foaming surfactant;
b) at least one nonionic cellulose selected from celluloses with at least one OH group substituted by a hydrophobic group; and
c) at least one oxidized and esterified starch.

### Foaming Surfactants

The composition according to the present invention comprises at least one foaming surfactant.

The surfactants may be selected from anionic, amphoteric (or zwitterionic), nonionic, cationic foaming surfactants, and mixtures thereof.

### Anionic Surfactants

The term anionic surfactant means a surfactant having only anionic groups as ionic or ionizable groups.

In the present description, an entity is qualified as “anionic” when it has at least one permanent negative charge or when it can be ionized by a negatively charged entity, under the conditions of use of the composition of the invention (medium, pH, for example) and containing no cationic charge.

The anionic surfactants may be sulfate(s) or sulfonate(s) which have at least one sulfate group (—OSO₃H or —OSO₃⁻), and/or a sulfonate group (—SO₃H or —SO₃), or else carboxylic or carboxylate surfactants having at least one carboxylic acid group (—COOH or —COO⁻).

It is understood that the anionic carboxylate surfactants may include one or more sulfate or sulfonate groups; sulfonate anionic surfactants may optionally further comprise one or more sulfate or carboxylate groups; and sulfate anionic surfactants may optionally further comprise one or more carboxylate or sulfonate groups.

Anionic surfactants which may be used include alkyl sulfates, alkyl ether sulfates, alkylamido ether sulfates, alkylaryl polyether sulfates, monoglyceride sulfates, alkyl sulfonates or alpha olefin sulfonates, alkylamide sulfonates, alkylarylsulfonates, paraffin sulfonates, alkyl sulfosuccinates, alkyl ether sulfo succinates, alkylamide sulfosuccinates, alkyl sulfo acetates, acylsarcosinates, acylglutamates, alkyl sulphosuccinamates, acylisethionates and N-acyl taurates, salts of alkyl monoesters and polyglycosidepolycarboxylic acids, acyl lactylates, N-acyl glycinates, salts of D-galactoside-uronic acids, salts of alkyl ether carboxylic acids, alkyl aryl ether carboxylic acid salts, salts of alkyl amidoether carboxylic acids, sulfoacetates, sulfolaurates, and the corresponding non-salt forms all of these compounds, the alkyl and acyl groups of all these compounds containing from 6 to 40 carbon atoms, especially 14 to 30 carbon atoms, more preferably from 16 to 22 carbon atoms; and aryl means phenyl group. These compounds may be ethoxylated and then preferably comprise from 1 to 50 ethylene oxide units.

Ethylene polyoxyalkylenated (C6-C24) (amido) ether carboxylic acids and salts thereof may also be cited, in particular those comprising from 2 to 50 alkylene oxide groups, in particular, such as sold by the company KAO under the names AKYPO.

The more preferred alkyl (C6-C24) (amido) ether carboxylic acids correspond to the following formula:

R₁—(OC₂H₄)n—OCH₂COOA (1)
wherein:
R₁ represents a radical or a mixture of linear or branched alkyl or alkenyl in C8-C22, a alkyl (C8-C9) phenyl radical, a R₂CONH—CH₂—CH₂— group with R₂ denoting an alkyl radical linear or branched alkenyl in C9-C21; preferably R₁ being an alkyl radical having 8 to 20 carbon atoms, preferably from 8 to 18 carbon atoms and aryl preferably denoting phenyl,
n is an integer or decimal number (average value) which may vary from 2 to 24 and preferably 2 to 10,
A denotes H, ammonium, Na, K, Li, Mg or monoethanolamine or triethanolamine.

It is also possible to use mixtures of compounds of formula (1), in particular mixtures in which the R₁ groups differ.

Polyoxyalkylenated (C6-C24) (amido) ether carboxylic acids preferably used in the present invention are selected from those of formula (1) wherein:
R₁ denotes a radical or a mixture of (C12-C14)alkyl radicals, cocoyl, oleyl, a nonyl or octylphenyl radical,
A denotes hydrogen or sodium, and
n is from 2 to 20 and preferably 2 to 10.

Polyoxy(C6-C24)alkylenated ether carboxylic acids and their salts are preferably used, and also polyoxyalkylenated (C6-C24)alkylamido ether carboxylic acids and salts thereof; in particular those having from 2 to 15 alkylene oxide groups.

Even more preferably, one can use the compounds of formula (1) wherein R₁ is a C12 alkyl radical, A denotes hydrogen or sodium and n is from 2 to 10.

Salts are especially selected from alkali metal salts, especially sodium, ammonium salts, amine salts, amino alcohol such as triethanolamine or monoethanolamine, and magnesium salts.

Preferably, the anionic surfactants are chosen from, alone or as a mixture:
(C6-C24)alkylsulfates, especially in C12-C20,
(C6-C24)alkyl ether sulfates, especially in C12-C20, preferably containing from 2 to 20 ethylene oxide units,
(C6-C24)alkylsulfosuccinates, especially in C12-C20, including laurylsulfosuccinates,
(C6-C24)alkyl ether sulfosuccinates, especially in C12-C20, (C6-C24)acyl sarcosinates, especially in C12-C20, including palmitoylsarcosinates,
(C6-C24)alkyl ether carboxylates, preferably (C12-C2o)alkyl ether carboxylates,
(C6-C24)acyl isethionates, preferably (Ci2-Cis)acyl isethionates,
polyoxyalkylenated (C6-C24)alkyl (amido) ether carboxylic acids and salts thereof, in particular those comprising from 2 to 50 alkylene oxide groups, especially ethylene,
(C6-C24)acylglutamates, especially in C12-C20,
(C6-C24)acylglycinates, especially in C12-C20,
particularly in the form of alkali or alkaline earth metal, ammonium, amine or aminoalcohol.

More Preferably, the anionic surfactant is chosen from (C6-C24)alkyl sulfates, (C6-C24)alkyl ether sulfates such as sodium lauryl ether sulfate, isethionates, amino acids, in particular glycinates, such as sodium. N-cocoyl glycinate, their alkali salts, and mixtures thereof.

### Amphoteric and Zwitterionic Foaming Surfactants

The amphoteric surfactants that may be used in the invention may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C8-C20)alkylbetaines, sulfobetaines, (C8-C20)alkylsulfobetaines, (C8-C20)alkylamido(C1-C(,)alkylbetaines, such as cocamidopropyibetaine, and (C8-C20)alkylamido(C1-C6)alkylsulfobetaines, and mixtures thereof.

Among the optionally quaternized secondary or tertiary aliphatic amine derivatives that may be used, mention may also be made of the products of respective structures (A1) and (A2) below:

Ra—CON(Z)CH₂—(CH₂)ₘ⁻N⁺(Rb)(Rc)(CH₂COO⁻) (A1)
in which:
Ra represents a (C10-C30)alkyl or alkenyl group derived from an acid Ra—COOH preferably present in hydrolyzed coconut oil, a heptyl group, a nonyl group or an undecyl group,
Rb represents a β-hydroxyethyl group,
Rc represents a carboxymethyl group,
m is equal to 0, 1 or 2,
Z represents a hydrogen atom or a hydroxyethyl or carboxymethyl group;

Ra′-—CON(Z)CH₂—(CH₂)ₘ⁻N(B)(B′) (A2)
in which:
B represents —CH₂CH₂OX′, with X′ representing —CH₂—COOH, CH₂—COOZ′, —CH₂CH₂—COOH, —CH₂CH₂—COOZ′, or a hydrogen atom,
B′ represents —(CH₂)_{z}—Y′, with z=1 or 2, and Y′ representing —COOH, —COOZ′, —CH₂—CHOH—SO₃H or —CH₂—CHOH—SO₃Z′,
m′ is equal to 0, 1 or 2,
Z represents a hydrogen atom or a hydroxyethyl or carboxymethyl group,
Z′ represents an ion resulting from an alkali or alkaline-earth metal, such as sodium, potassium or magnesium; an ammonium ion; or an ion resulting from an organic amine and in particular from an amino alcohol, such as monoeihanolamine, diethanolamine and triethanolamine, monoisopropanolamine, diisopropanolamine or triisopropanolamine, 2-ammo-2-methyl-1-propanol, 2-amino-2-methyl-1,3-propanediol and tris(hydroxymemyl)aminomethane,
Ra′ represents a (C10-C30)alkyl or alkenyl group of an acid Ra′COOH preferably present in hydrolyzed linseed oil or coconut oil, an alkyl group, in particular a C17 alkyl group, and its iso form, or. an unsaturated C17 group.

The compounds corresponding to formula (A2) are preferred.

Among the compounds corresponding to formula (A2) in which X′ represents an hydrogen atom, mention may be made of compounds classified in the CTFA dictionary, under the names sodium cocoampho acetate, sodium lauroamphoacetate, sodium caproamphoacetate and sodium capryloamphoacetate.

Compounds corresponding to formula (A2) may be (C8-C20) alkylamphoacetates and (C8-C20)alkylamphodiacetates and mixtures thereof.

Other compounds corresponding to formula (A2) are disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryioamphodipropionate, lauroamphodipropionic acid and cocoamphodipropionic acid.

Examples that may be mentioned include the cocoamphodiacetate sold by the company Rhodia under the trade name Miranol® C2M Concentrate, the sodium cocoamphoacetate sold under the trade name Miranol Ultra C32 and the product sold by the company Chimex under the trade name Chimexane HA.

Use may also be made of the compounds of formula (A3):

Ra″—NH—CH(Y″)—(CH₂)ₙ—C(O)—NH—(CH₂)n′—N(Rd)(Re) (A3)
in which:
Ra″ represents a (C10-C30)alkyl or alkenyl group of an acid Ra″—C(O)OH preferably present in hydrolysed linseed oil or coconut oil;
Y″ represents the group —C(O)OH, —C(O)OZ″, —CH₂—CH(OH)—SO₃H or the group —CH₂—CH(OH)—SO₃—Z″, with Z″ representing a cationic counterion resulting from an alkali metal or alkaline-earth metal, such as sodium, an ammonium ion or an ion resulting from an organic amine;
Rd and Re represent, independently of each other, a (C1-C4)alkyl or hydroxyalkyl radical; and
n and n′ denote, independently of each other, an integer ranging from 1 to 3.

Among the compounds corresponding to formula (A3), mention may in particular be made of the compound classified in the CTFA dictionary under the name sodium diethylaminopropyl cocoaspartamide, such as the one sold by the company Chimex under the name Chimexane HB.

Preferably, the amphoteric surfactants are chosen from (C8-C20)alkylbetames, (C8-C20)alkylamido(C1-C6)alkylbetaines, (C8-C20)alkylamphoacetates and (C8-C20)alkylamphodiacetates, and mixtures thereof.

### Nonionic Surfactants

Nonionic surfactant can be nonionic alkylpolyglycoside surfactants, especially represented by formula (I):

R₁O—(R₂O)t—(G)v (I)
wherein:
R₁ represents a linear or branched alkyl or alkenyl radical having 6 to 24 carbon atoms, especially 8 to 18 carbon atoms, or an alkylphenyl radical whose linear or branched alkyl radical comprises from 6 to 24 carbon atoms, especially 8 to 18 carbon atoms,
R₂ represents an alkylene radical having 2 to 4 carbon atoms,
G is a sugar unit containing 5 to 6 carbon atoms,
1 is a value ranging from 0 to 10, preferably from 0 to 4,
v is a value ranging from 1 to 15, preferably from 1 to 4.

Preferably, the alkylpolyglycoside surfactants are compounds of formula (I) described above wherein:
R₁ denotes a linear or branched saturated or unsaturated alkyl radical having 8 to 18 carbon atoms,
R₂ represents an alkylene radical having 2 to 4 carbon atoms,
t is a value ranging from 0 to 3, preferably equal to 0,
G denotes glucose, fructose or galactose, preferably glucose,
the degree of polymerization, ie the value of v can range from 1 to 15, preferably from 1 to 4; the average degree of polymerization is more particularly between 1 and 2.

Glycosidic linkages between the sugar units are generally 1-6 or 1-4, preferably 1-4.

Preferably, the alkylpolyglycoside surfactant is an alkylpolyglucoside surfactant, even more preferably an C8-C16 alkylpolyglucosides, and particularly preferably chosen among decylglucosides, caprylyl/capryl glucosides, laurylglucoside, cocoylglucoside, caprylyiglucoside, and mixtures thereof.

Among the commercial products, the following product may be cited: products sold by COGN1S under the names PLANTAREN® (600 CS/U, 1200 and 2000) or PLANTACARE® (818, 1200 and 2000); products sold by SEPPIC under the names ORAMIX® CG 11.0 and ORAMIX® NS 10; products sold by BASF under the name LUTENSOL GD 70 or products sold by the company CHEM Y under the name AGIO LK.

Preferably, C8-C16 alkylpolyglucosides is used, in particular chosen from decylglucoside, caprylyl/capryl glucoside, laurylglucoside, cocoylglucoside, caprylylglucoside, and mixtures thereof.

### Cationic Surfactants

They are advantageously chosen from optionally polyoxyalkylenated primary, secondary or tertiary fatty amine salts, quaternary ammonium salts, and mixtures thereof.

Mention may be made in particular of:
quaternary ammonium salts having formula (Ia):

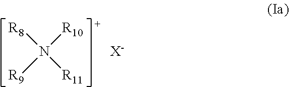

in which:
the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ containing from 8 to 30 carbon atoms and. preferably from 12 to 24 carbon atoms; it being possible for the aliphatic groups to comprise heteroatoms such as, in particular, oxygen, nitrogen, sulfur or halogens; and
X⁻ is an anion that could be chosen from the group consisting of halides, phosphates, acetates, lactates, (C1-C4)alkyl sulfates, (C1-C4)alkylsulfonates and (C1-C4)alkylarylsulfonates.

The aliphatic groups are chosen, for example, from (C1-C30)alkyl, (C1-C30)alkoxy, (C2-C6)polyoxyalkylene, (C1-C30)alkylamide, (C12-C22)alkyl(C2-C6)alkylamido, (C12-C22)alkyl acetate and hydroxy(C1-C30)alkyl groups.

Mention may be made of tetraalkylammonium halides, preferably chlorides, such as dialkyldimethylammonium or alkyltrimethylanmionium chlorides in which the alkyl group comprises from 12 to 22 carbon atoms, particularly behenyltrimethylammonium, distearyldimethylammonium, cetyltrimethylammonium, and benzyldimethylstearylammonium chlorides.

Mention may also be made of palmitylamidopropyltrimethylammonium halides, preferably chlorides, or stearamidopropyldimethyl(myristyl acetate)-ammonium halides, preferably chlorides, such as the product sold under the name Ceraphyl® 70 by the company VAN DYK.
imidazoline quaternary ammonium salts having formula (Ila):

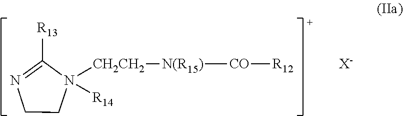

in which:
R₁₂ represents an alkenyl or alkyl group comprising from 8 to 30 carbon atoms, for example fatty acid derivatives of tallow;
R₁₃ represents a hydrogen atom, a (C1-C4)alkyl group or an alkenyl or alkyl group comprising from. 8 to 30 carbon atoms;
R₁₄ represents a (C1-C4)alkyl group;
R₁₅ represents a hydrogen atom or a (C1-C4)alkyl group;
X⁻ is an anion, preferably chosen from the group consisting of halides, phosphates, acetates, lactates, (C1-C4)alkyl sulfates, (C1-C4)alkylsulfonates and (C1-C4)alkylarylsulfonates;
R₁₂ and R₁₃ preferably denote a mixture of alkenyl or alkyl groups containing from 12 to 21 carbon atoms, for example fatty acid derivatives of tallow, R₁₄ denotes a methyl group, and R₁₅ denotes a hydrogen atom. A product of this kind is sold for example under the name Rewoquat® W 75 by the company Rewo.
quaternary di- or triammonium salts having formula (Illa):

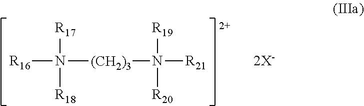

in which:
R₁₆ denotes an alkyl group containing from 16 to 30 carbon atoms, which is optionally hydroxylated and/or interrupted with one or more oxygen atoms,
R₁₇ represents a hydrogen atom or an alkyl group containing from 1 to 4 carbon atoms or a group —(CH₂)₃—N⁺(R₁₆ₐ)(R₁₇ₐ)(R₁₈ₐ), wherein R₁₆ₐ, R₁₇ₐ, R₁₈ₐ, which may be identical or different, are chosen from hydrogen and an alkyl group comprising from 1 to 4 carbon atoms
R₁₈, R₁₉, R₂₀ and R₂₁, which may be identical or different, are chosen from hydrogen and an alkyl group comprising from 1 to 4 carbon atoms; and
X⁻ is an anion preferably chosen from the group consisting of halides, acetates, phosphates, nitrates, (C1-C4) alkyl sulfates, (C1-C4) alky Isulfonates and (C1-C4)alkylarylsulfonates, in particular methyl sulfate and ethyl sulfate.

Such compounds are, for example, Finquat CT-P, sold by the company Finetex (Quatemium 89), and Finquat CT, sold by the company Finetex (Quatemium 75).
quaternary ammonium salts containing one or more ester functions having following formula (IVa):

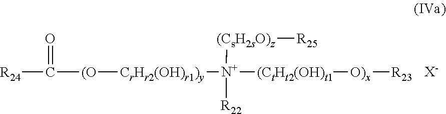

in which:
R₂₂ is chosen from (C1-C6)alkyl groups and hydroxy(C1-C6)alkyl or dihydroxy(C1-C6)alkyl groups;
R₂₃ is chosen from the group R₂₆—C(═O)—; hydrocarbon-based linear or branched, saturated or unsaturated C1-C22groups R₂₇; and a hydrogen atom;
R₂₅ is chosen from the group R₂₈—C(═O)—; hydrocarbon-based linear or branched, saturated or unsaturated C1-C6 groups R₂₉; and a hydrogen atom;
R₂₄, R₂₆ and R₂₈, which may be identical or different, are chosen from linear or branched, saturated or unsaturated C7-C21 hydrocarbon-based groups;
r, s and t, which may be identical or different, are integers ranging from 2 to 6;
r1 and t1, which may be identical or different, are equal to 0 or 1;
r2+r1=2r and t1+t2=2t;
y is an integer ranging from 1 to 10;
x and z, which may be identical or different, are integers ranging from 0 to 10; the sum x+y+z is from 1 to 15,
X⁻ is an anion;
with the proviso that, when x is 0 then R₂₃ denotes R₂₇, and that when z is 0 then R₂₅ denotes R₂₉.

The alkyl groups R₂₂ may be linear or branched, and more particularly linear. Preferably, R₂₂ denotes a methyl, ethyl, hydroxyethyl or dihydroxypropyl group, and more particularly a methyl or ethyl group.

When R₂₃ is an R₂₇ hydrocarbon group, it may have from 12 to 22 carbon atoms, or may have from 1 to 3 carbon atoms.

When R₂₅ is an R₂₉ hydrocarbon group, it preferably has 1 to 3 carbon atoms.

Advantageously, R₂₄, R₂₆ and R₂₈, which are identical or different, are chosen from linear or branched, saturated or unsaturated C11-C21 hydrocarbon groups, and more particularly from linear or branched C11-C21 alkyl and alkenyl groups.

Preferably, x and z, which may be identical or different, are equal to 0 or 1. Advantageously, y is equal to 1.

Advantageously, the sum x+y+z is from 1 to 10.

Preferably, r, s and t, which may be identical or different, are equal to 2 or 3, and even more particularly are equal to 2.

The anion X⁻ is preferably a halide, preferably chloride, bromide or iodide, a (C1-C4)alkyl sulfate, (C1-C4)alkyl sulfonate or (C1-C4)alkylaryl sulfonate, methanesulfonate, phosphate, nitrate, tosylate, an anion derived from an organic acid, such as acetate or lactate, or any other anion that is compatible with the ammonium comprising an ester function. The anion X is more particularly chloride, methyl sulfate or ethyl sulfate.

Use is made more particularly, in the composition according to the invention, of the ammonium salts having formula (IVa) in which:
R₂₂ denotes a methyl or ethyl group,
x and y are equal to 1,
z is equal to 0 or 1,
r, s and t are equal to 2,
R₂₃ is chosen from the group R₂₆—C(═O)—; methyl groups, ethyl groups or hydrocarbon-based C14-C22 groups; and a hydrogen atom,
R₂₅ is chosen from the group R₂₈—C(═O)—; and a hydrogen atom,
R₂₄, R₂₆ and R₂₆, which may be identical or different, are chosen from linear or branched, saturated or unsaturated C13-C17 hydrocarbon groups, and preferably from linear or branched, saturated or unsaturated C13-C17 alkyl and alkenyl groups.

The hydrocarbon-based groups are advantageously linear.

Among the compounds of formula (IVa), examples that may be mentioned include salts, in particular the chloride or methyl sulfate of diacyloxyethyldimethylammonium, diacyloxyethylhydroxyethylmethylammonium, monoacyloxyethyldibydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent, such as an alkyl halide, preferably methyl or ethyl halide, a dialkyl sulfate, preferably methyl or ethyl sulfate, methyl methanesulfonate, methyl para-toluenesulfonate, glycol chlorohydrin or glycerol chlorohydrin. Such compounds are, for example, sold under the names Dehyquart® by the company Henkel, Stepanquat® by the company Stepam Noxamium® by the company Ceca or Rewoquat® WE 18 by the company Rewo-Witco.

The composition according to the invention may contain, for example, a mixture of quaternary ammonium monoester, diester and triester salts with a weight majority of diester salts. Use may also be made of the ammonium salts containing at least one ester function that are described in patents US-A-4 874 554 and US-A-4 137 180. Use may also be made of behenoylhydroxypropyltrimethylammonium chloride, for example, sold by the company Kao under the name Quartamin BTC 131.

Preferably, the ammonium salts containing at least one ester function contain two ester functions.

Preferably, the cationic surfactants are chosen from the compounds of formula (Ia) and the compounds of formula (IVa), preferably from cetyltrimethylammonium, behenyltrimethylammonium, and dipalmitoylethyihydroxyethylmethylammonium salts, and mixtures thereof, and more particularly from behenyltrimethylammonium chloride or methosulfate, cetyltrimethylammonium chloride or methosulfate, and dipalmitoylethylhydroxyethylammonium chloride or methosulfate, and mixtures thereof. Even more preferentially, the cationic surfactant is a behenyltrimethylammonium salt.

According to a preferred embodiment, the composition comprises at least one anionic surfactant, preferably chosen from alkyl ether sulfate comprising 6 to 24 carbon atoms, preferably 2 to 20 carbon atoms; amino acids, in particular glycinates; and mixtures thereof.

In a particular embodiment, the composition comprises at least one anionic foaming surfactant, preferably chosen from sodium lauroyl sarcosinate, sodium cocoyl glycinate, sodium cocoyl glutamate, sodium stearoyl glutamate, disodium cocoyl glutamate, sodium lauroyl glutamate, potassium cocoyl glycinate, TEA-cocoyl glutamate, or a mixture thereof.

Advantageously, the foaming surfactant is present in the composition according to the present invention in an amount ranging from 1 wt. % to 60 wt. %, preferably from 20 wt. % to 50 wt. %, more preferably from 35 wt. % to 45 wt. %, relative to the total weight of the composition.

### Nonionic Celluloses

The composition according to the present invention comprises at least one nonionic cellulose selected from celluloses with at least one OH group substituted by a hydrophobic group.

As examples for hydrophobic groups, mention can be made of C1-C4 alkyls, hydroxy C1-C4 alkyls, and carboxy C1-C4 alkyls.

Preferably, the nonionic cellulose is selected from methylcellulose, hydroxymethyl methylcellulose, hydroxyethyl methylcellulose, hydroxypropyl methylcellulose, carboxymethyl methylcellulose, carboxyethyl methylcellulose, carboxypropyl methylcellulose, mixtures thereof.

The nonionic celluloses that are suitable for use in the invention have a mass-average molecular weight of between 1000 and 10 000 000 g/mol, preferably between 20 000 and 5 000 000 g/mol and even more preferentially between 50 000 and 3 000 000 g/mol.

As commercial products of nonionic cellulose suitable for the composition according to the present invention, mention can be made of hydroxypropyl methylcellulose sold under the name METHOCEL™ E 4 M PREMIUM by the company DOW.

Advantageously, the nonionic cellulose is present in the composition according to the present invention in an amount ranging from 0.03 wt. % to 3 wt. %, preferably from 0.05 wt. % to 0.2 wt. %, more preferably from 0.1 wt. % to 0.15 wt. %, relative to the total weight of the composition.

### Oxidized and Esterified Starches

The composition according to the present invention comprises at least one oxidized and esterified starch (also called as oxidized starch ester).

The term “oxidized” means that at least one hydroxyl of the starch has been oxidized, in particular in position 6 of a glucose unit of the starch: the groups —CH₂—OH becoming an aldehyde group —C(O)—H or carboxylic group —C(O)—OH.

The term “ester” or “esterified” means that at least one hydroxyl group of the glucose unit A of the starch is esterified with a (thio)carboxylic acid, particularly a (C1-C8)(thio)carboxylic acid, and preferably with an acetic acid to obtain an ester B, C or D with C and D corresponding, respectively, to the end or the start of the starch, Ra, Rb, Rc, Rd and Re representing a hydrogen atom or an alkyl(thio)carbonyl group such as (C1-C8)alkyl-C(O)— or (C1-C8)alkyl-C(S)—, it being understood that at least one of the radicals Ra, Rb, Rc, Rd and Re represents an alkyl(thio)carbonyl in at least one unit of the starch:

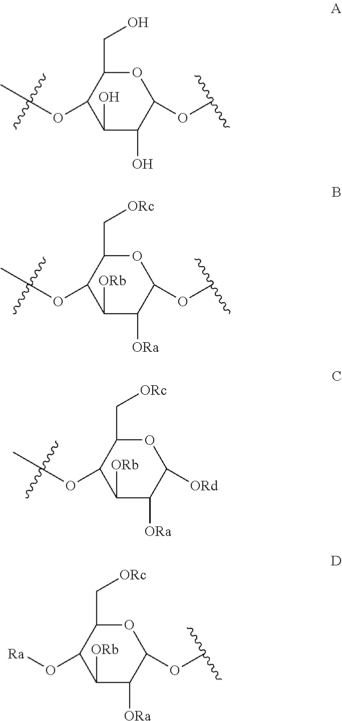

Thus, “oxidized ester” means that at least one hydroxyl group of the starch is oxidized as defined above and that at least one other hydroxyl group of the starch is esterified as defined above.

According to one embodiment, the composition of the invention comprises a starch which comprises at least one linear glucose polymer, i.e. an amylose derivative of formula (a) and/or at least one branched glucose polymer, i.e. an amylopectin derivative of formula (b), anomers thereof, preferably alpha-anomers, salts thereof and solvates thereof such as hydrates thereof:

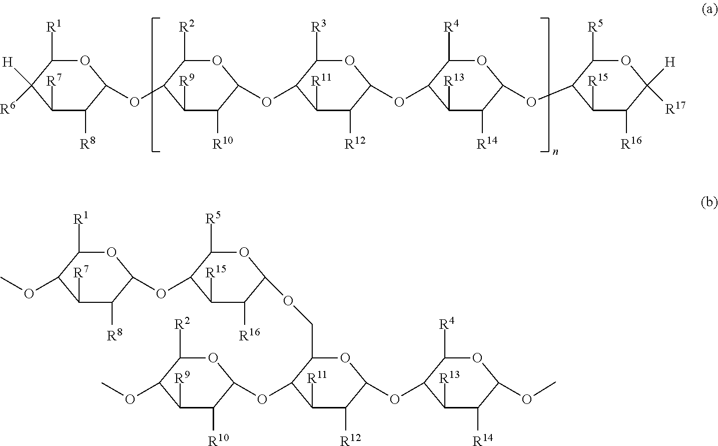

in which formulae (a) and (b):
R₁, R₂, R₃, R₄ and R₅, which may be identical or different, represent a hydroxymethyl group —CH₂—OH or an oxidized form such as —CH₂—C(O)—H, or —C(O)—OH, it being understood that at least one of the radicals R₁, R₂, R₃, R₄ or R represents an oxidized form, preferably —C(O)—OH, and more particularly R is an oxidized form and R₁, R₂, R₃ and R₅ represents a hydroxymethyl group;
R₇, R₈, R₉, R₁₀, R₁₁, R₁₂, R₁₃, R₁₄, R₁₅, and R₁₆, which may be identical or different, represent a hydroxyl group or a group (C1-C8)alkylcarbonyloxy, or a group (C1-C8)alkylthiocarbonyloxy, preferably a hydroxyl group or a group (C1-C6)alkylcarbonyloxy such as an acetyl group, it being understood that at least one of the radicals R₇, R₈, R₉, R₁₀, R₁₁, R₁₂, R₁₃, R₁₄, R₁₅ or R₁₆ represents a group (C1-C8)alkylcarbonyloxy, or a group (C1-C8)alkylthiocarbonyloxy, preferably a group (C1-C6)alkylcarbonyloxy such as an acetyl group, more particularly, R₇, R₈, R₉, R₁₀, R₁₁, R₁₃, R₁₄, R₁₅ and R₁₆ represent a hydroxyl group and R₁₂ represents a group (C1-C8) alkylcarbonyloxy or a group (C1-C8)alkylthiocarbonyloxy, preferably a group (C1-C8)alkylcarbonyloxy such as an acetyl group
n represents an integer, for example between 50 and 300 000, preferably between 100 and 200 000, more particularly from 200 to 100 000, better still from 400 to 100 000 and even better still from 470 to 95 000.

The average molecular weight of the modified amylopectin of formula (b) is at least 10⁷ and may be greater than 10⁹.

According to one preferred embodiment of the invention, the oxidized starch ester comprises a mixture of linear and branched glucose polymers, i.e. a mixture of amylose derivatives and of amylopectin derivatives and more particularly in an amount ranging from 10% to 40% by weight of amylose and from 60% to 90% of amylopectin; and preferably from 15% to 20% of amylose and from 80% to 85% of amylopectin.

According to another preferred embodiment of the invention, the oxidized starch ester comprises a major amount of amylose derivatives, preferably from 60% to 100% and more particularly from 80% to 85% by weight.

According to a preferred embodiment of the invention, the oxidized starch ester, preferably oxidized starch acetate, comprises amylose derivatives of formula (a) with an average molecular weight of 10⁴ to 10⁷, preferably 10⁵ to 10⁶, which means that they have a mean degree of polymerization (DP, number of glucosyl units per molecule) ranging from 50 to 20 000, preferably from 100 to 10 000 and more particularly from 600 to 6000.

The esterification index is preferably greater than 0.05.

The oxidized starch ester may be readily produced in a manner known to those skilled in the art. For example, the manufacturing process includes two reaction steps of oxidation and esterification.

As commercial products of oxidized starch acetate suitable for the composition according to the present invention, mention can be made of that sold under the name GF-A390 by the company FOSHAN GAOFENG STARCH TECHNOLOGY.

Advantageously, the oxidized and esterified starch is present in the composition according to the present invention in an amount ranging from 0.3 wt. % to 3 wt. %, preferably from 0.5 wt. % to 2 wt. %, more preferably from 1 wt. % to 1.5 wt. %, relative to the total weight of the composition.

Advantageously, the weight ratio of the nonionic cellulose selected from methylcelluloses optionally substituted by a hydroxy C1-C4 alkyl or a carboxy C1-C4 alkyl to the oxidized and esterified starch ranges from 1:10 to 10:1, preferably 1:3 to 3:1, more preferably 1: 1.5 to 1.5:1.

### Aqueous Phase

The composition of the present invention comprises at least one aqueous phase.

According to a preferred embodiment, the composition of the present invention is an aqueous paste.

The aqueous phase of the composition according to the present invention comprises water and optionally one or more water-miscible or at least partially water-miscible compounds, for instance C2-C8 polyols or monoalcohols, such as ethanol and isopropanol.

The term “polyol” should be understood as meaning any organic molecule comprising at least two free hydroxyl groups. Examples of polyols that may be mentioned include glycols, for instance butylene glycol, propylene glycol, dipropylene glycol, isoprene glycol, hexylene glycol, caprylyl glycol, glycerol (i.e. glycerin) and polyethylene glycols.

According to a preferred embodiment, the composition according to the present application further comprises at least one C2-C8 polyol selected from butylene glycol, propylene glycol, dipropylene glcol, isoprene glycol, hexylene glycol, caprylyl glycol, glycerin and polyethylene glycols. More preferably, the C2-C8 polyol is glycerin.

Preferably, the C2-C8 polyol is present in an amount ranging from 1 wt. % to 70 wt. %, preferably from 30 wt. % to 40 wt %, relative to the total weight of the composition.

The aqueous phase may represent from 40 wt. % to 70 wt. %, preferably from 45 wt. % to 65 wt. %, relative to the total weight of the composition.

### Additional Ingredients

The composition according to the present invention may comprise one or more additional ingredients, selected from those conventionally used in skin cleanser.

The composition in accordance with the present invention may comprise any of the following additives: pH adjusting agents (e.g. citric acid); additional surfactants; biological extracts; antibacterial agents, fragrances; thickeners (such as acrylates copolymer); structuring agents (such as potassium chloride, cetyl alcohol), and cationic preservatives.

A person skilled in the art can adjust the type and amount of additional ingredients present in the compositions according to the present invention by means of routine operations, so that the desired properties of these compositions are not adversely affected by the additional ingredients.

According to a preferred embodiment, the present invention relates to a composition for cleansing and/or removing makeups from keratin materials, comprising, relative to the total weight of the composition:
a) from 35 wt. % to 45 wt. % of at least one foaming surfactant;
b) from 0.1 wt. % to 0.15 wt. % of at least one nonionic cellulose selected from methylcellulose and hydroxypropyl methylcellulose; and
c) from 1 wt. % to 1.5 wt. % of at least one oxidized starch acetate.

The composition according to the present invention may comprise an oil. Preferably, the composition according to the present invention does not comprise any oil.

### Process and Use

The composition according to the present invention can be used in a process for cleansing and/or removing makeups from keratin materials, such as the skin, in particular the face, by being applied to the keratin materials.

The composition according to the present invention can be a crystalized system in the form of a paste, a cream or a gel textured lotion.

It was found that the composition according to the present invention can provides a good foam density.

The composition according to the invention may be applied by any means enabling a uniform distribution, in particular using a finger, or a cotton ball, and can be removed by rinsing with water.

Thus, according to another aspect, the present invention relates to a non-therapeutic process for cleansing and/or removing makeups from keratin materials, in particular the skin, comprising the application to the keratin materials, in particular the skin, of the composition according to the present invention, and rinsing off said composition after an optional period of time.

The present invention is illustrated in greater detail by the examples described below, which are given as non-limiting illustrations.

### EXAMPLES

Main raw materials used, trade names and supplier thereof are listed in Table 1.

TABLE 1

| INCI Name | Trade Name | Supplier |
|---|---|---|
| HYDROXYPROPYL | METHOCEL ™ E 4 M PREMIUM | DOW |
| METHYLCELLULOSE | | |
| OXIDIZED STARCH ACETATE | GF-A390 | FOSHAN GAOFENG |
| | | STARCH TECHNOLOGY |
| SODIUM COCOYL GLYCINATE | AMILITE ® GCS-12K | AJINOMOTO |
| GLYCERIN | GLICENAT GC K MB | OXITENO |
| POTASSIUM CHLORIDE | CLORETO DE POTASSIO | LABSYNTH |
| | U.S.P/F.C.C | |
| CETYL ALCOHOL | LANETTE 16 | BASF |
| CITRIC ACID | CITRIC ACID MONOHYDRATE | CITRIQUE BELGE |
| | GRANULAR | |
| ACRYLATES COPOLYMER | CARBOPOL ® AQUA SF-1 | LUBRIZOL |
| | POLYMER | |
| HYDROXYETHYLCELLULOSE | NATROSOL ™ 250 HHR CS | ASHLAND |
| ZEA MAYS (CORN) STARCH | BEAUTE-BY-ROQUETTE ® | ROQUETTE |
| | ST005 | |

### Invention Examples 1-5 and Comparative Example 1-5

Compositions according to invention examples (IE) 1-5 and comparative examples (CE) 1-5 comprising the ingredients shown in Table 2 were prepared, with all amounts expressed by percentages by weight of active matter with regard to the total weight of each composition.

TABLE 2

| Components | IE.1 | IE.2 | IE.3 | IE.4 | IE.5 | CE.1 | CE.2 | CE.3 | CE.4 | CE.5 |
|---|---|---|---|---|---|---|---|---|---|---|
| Sodium cocoyl | 40 | 40 | 40 | 40 | 40 | 40 | 40 | 40 | 40 | 40 |
| glycinate | | | | | | | | | | |
| Glycerin | 35 | 35 | 35 | 35 | 35 | 35 | 35 | 35 | 35 | 35 |
| Water | QS | QS | QS | QS | QS | QS | QS | QS | QS | QS |
| Hydroxypropyl | 0.1 | 0.05 | 0.2 | 0.1 | 0.1 | — | — | 0.1 | — | 0.1 |
| methylcellulose | | | | | | | | | | |
| Hydroxyethylcellulose | — | — | — | — | — | — | — | — | 0.1 | — |
| Oxidized starch acetate | 1 | 1 | 1 | 0.5 | 2 | — | 1 | — | 1 | — |
| Zea mays (corn) starch | — | — | — | — | — | — | — | — | — | 1 |
| Acrylates copolymer | 5 | 5 | 5 | 5 | 5 | 5 | 5 | 5 | 5 | 5 |
| Potassium chloride | 2 | 2 | 2 | 2 | 2 | 2 | 2 | 2 | 2 | 2 |
| Cetyl alcohol | 2 | 2 | 2 | 2 | 2 | 2 | 2 | 2 | 2 | 2 |
| Citric acid | 0.6 | 0.6 | 0.6 | 0.6 | 0.6 | 0.6 | 0.6 | 0.6 | 0.6 | 0.6 |

Compositions of Invention examples 1-5 belong to the present invention.

Composition of comparative example 1 does not comprise an optionally substituted methylcellulose and an oxidized starch ester.

Composition of comparative example 2 does not comprise a celluloses with at least one OH group substituted by a hydrophobic group.

Composition of comparative example 3 does not comprise an oxidized starch ester.

Composition of comparative example 4 comprises hydroxyethylcellulose instead of a cellulose with at least one OH group substituted by a hydrophobic group.

Composition of comparative example 5 comprises zea mays (corn) starch instead of an oxidized starch ester.

### Preparation Process

The compositions were prepared as follows, taking the composition of invention example 1 as an example.

Glycerin and sodium cocoyl glycinate were added into a main beaker, followed by hydroxypropyl methylcellulose, oxidized starch acetate and acrylates copolymer with stirring and heating to 70-85° C. Next, potassium chloride and cetyl alcohol were added at a temperature higher than 70° C., then citric acid and water were added to obtain the composition. The composition was cooled down to 30° C. and aged for 24 hours.

### Evaluation of Compositions

The compositions prepared were evaluated on the foam density.

### Foam Density

Foam density was evaluated by observing the foam generated via hands as follows.

### Procedure:

1) Wet hands with nitrile gloves and apply 0.5 g of the composition to be tested onto the glove;
2) Rub the composition with two palms for 60 rounds (2 circles/second); and
3) Put together all the foam in one palm.

A score within a range of 1-15 was given, wherein 1-4 means a low foam density, 5-9 means a middle foam density and 10-15 means a high foam density.

The results obtained were summarized in Table 3.

TABLE 3

| Properties | IE.1 | IE.2 | IE.3 | IE.4 | IE.5 | CE.1 | CE.2 | CE.3 | CE.4 | CE.5 |
|---|---|---|---|---|---|---|---|---|---|---|
| Foam density | 14 | 11 | 12 | 11 | 13 | 5 | 9 | 3 | 1 | 1 |

It can be seen from Table 3, as compared with compositions of comparative examples 1-5, compositions of invention examples 1-5 show better foam density.

It was also found that the compositions of invention examples 1-5 do not have obvious change in appearance for 2 months at a temperature of 4° C. and 45° C.

1. A composition for cleansing and/or removing makeups from keratin materials, comprising:
a) at least one foaming surfactant;
b) at least one nonionic cellulose selected from celluloses with at least one OH group substituted by a hydrophobic group; and
c) at least one oxidized and esterified starch.

2. The composition according to claim 1, wherein the at least one foaming surfactant is selected from the group consisting of anionic, amphoteric, zwitterionic, nonionic, cationic foaming surfactants, and mixtures thereof.

3. The composition according to claim 1, wherein the composition comprises at least one anionic foaming surfactant.

4. The composition according to claim 1, wherein the at least one foaming surfactant is present in an amount ranging from 1 wt. % to 60 wt. % relative to the total weight of the composition.

5. The composition according to claim 1, wherein the at least one nonionic cellulose is selected from the group consisting of methylcellulose, hydroxymethyl methylcellulose, hydroxyethyl methylcellulose, hydroxypropyl methylcellulose, carboxymethyl methylcellulose, carboxyethyl methylcellulose, carboxypropyl methylcellulose, and mixtures thereof.

6. The composition according to claim 1, wherein the at least one nonionic cellulose is present in an amount ranging from 0.03 wt. % to 3 wt. % relative to the total weight of the composition.

7. The composition according to claim 1, wherein at least one hydroxyl group of the glucose unit of the at least one oxidized and esterified starch is esterified with a (thio)carboxylic acid.

8. The composition according to claim 1, wherein the at least one oxidized and esterified starch is oxidized starch acetate.

9. The composition according to claim 1, wherein the oxidized and esterified starch is present in an amount ranging from 0.3 wt. % to 3 wt. % relative to the total weight of the composition.

10. The composition according to claim 9, wherein the weight ratio of the nonionic cellulose to the oxidized and esterified starch ranges from 1:10 to 10:1.

11. The composition according to claim 1, further comprising at least one C2-C8 polyol selected from the group consisting of butylene glycol, propylene glycol, dipropylene glycol, isoprene glycol, hexylene glycol, caprylyl glycol, glycerin and polyethylene glycols.

12. The composition according to claim 11, wherein the C2-C8 polyol is present in an amount ranging from 1 wt. % to 70 wt. % relative to the total weight of the composition.

13. The composition according to claim 1, wherein the aqueous phase represents from 40 wt. % to 70 wt. % relative to the total weight of the composition.

14. The composition according to claim 1, comprising, relative to the total weight of the composition:
a) from 35 wt. % to 45 wt. % of at least one foaming surfactant;
b) from 0.1 wt. % to 0.15 wt. % of at least one nonionic cellulose selected from methylcellulose and hydroxypropyl methylcellulose; and
c) from 1 wt. % to 1.5 wt. % of at least one oxidized starch acetate.

15. A non-therapeutic process for cleansing and/or removing makeups from keratin materials, comprising the application to the keratin materials, of the composition according to claim 1, and rinsing off said composition after an optional period of time.
